SEQUENCE LISTING (2 sequences)

SEQ ID NO: 1 - DNA, 1200 bp, Homo sapiens
atggctcaaaggcagcctcactcacctaatcagactctaatttcaatcacaaatgacacagaatcatcaagctctgtggtttctaacgacaacacgaataaaggatggagcggggacaactctccaggaatagaagcattgtgtgccatctatattacttatgctgtgatcatttcagtgggcatccttggaaatgctattctcatcaaagtctttttcaagaccaaatccatgcaaacagttccaaatattttcatcaccagcctggcttttggagatcttttacttctgctaacttgtgtgccagtggatgcaactcactaccttgcagaaggatggctgttcggaagaattggttgtaaggtgctctctttcatccggctcacttctgttggtgtgtcagtgttcacattagcaattctcagcgctgacagatacaaggcagttgtgaagccacttgagcgacagccctccaatgccatcctgaagacttgtgtaaaagctggctgcgtctggatcgtgtctatgatatttgctctacctgaggctatattttcaaatgtatacacttttcgagatcccaataaaaatatgacatttgaatcatgtacctcttatcctgtctctaagaagctcttgcaagaaatacattctctgctgtgcttcttagtgttctacattattccactctctattatctctgtctactattccttgattgctaggaccctttacaaaagcaccctgaacatacctactgaggaacaaagccatgcccgtaagcagattgaatcccgaaagagaattgccagaacggtattggtgttggtggctctgtttgccctctgctggttgccaaatcacctcctgtacctctaccattcattcacttctcaaacctatgtagacccctctgccatgcatttcattttcaccattttctctcgggttttggctttcagcaattcttgcgtaaacccctttgctctctactggctgagcaaaagcttccagaagcattttaaagctcagttgttctgttgcaaggcggagcggcctgagcctcctgttgctgacacctctcttaccaccctggctgtgatgggaacggtcccgggcactgggagcatacagatgtctgaaattagtgtgacctcgttcactgggtgtagtgtgaagcaggcagaggacagattctag

SEQ ID NO: 2 - protein, 399 aa, Homo sapiens
MAQRQPHSPNQTLISITNDTESSSSVVSNDNTNKGWSGDNSPGIEALCAIYITYAVIISVGILGNAILIKVFFKTKSMQTVPNIFITSLAFGDLLLLLTCVPVDATHYLAEGWLFGRIGCKVLSFIRLTSVGVSVFTLAILSADRYKAVVKPLERQPSNAILKTCVKAGCVWIVSMIFALPEAIFSNVYTFRDPNKNMTFESCTSYPVSKKLLQEIHSLLCFLVFYIIPLSIISVYYSLIARTLYKSTLNIPTEEQSHARKQIESRKRIARTVLVLVALFALCWLPNHLLYLYHSFTSQTYVDPSAMHFIFTIFSRVLAFSNSCVNPFALYWLSKSFQKHFKAQLFCCKAERPEPPVADTSLTTLAVMGTVPGTGSIQMSEISVTSFTGCSVKQAEDRF

(19) United States
(12) Patent Application Publication
(10) Pub. No.: US 2004/0023862 A1
(43) Pub. Date: Feb. 5, 2004

(54) Uses of bombesin receptor 3

(75) Inventors: Darren Smart, Harlow (GB); Paul J L M Strijbos, Harlow (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(73) Assignee: SmithKline Beedham p. l. c., Brentford Middlesex TW8 9GS (GB)

(21) Appl. No.: 10/221,384

(22) PCT Filed: Mar. 13, 2001
(86) PCT No.: PCT/EP01/02812

(30) Foreign Application Priority Data
Mar. 15, 2000 (GB) 0006289.3

(51) Int. Cl. A61K 38/17; A61K 48/00

(52) U.S. Cl. 514/12; 514/44

(57) ABSTRACT

The invention relates to newly identified uses of BRS3 polypeptides and polynucleotides encoding such polypeptides, to their use in therapy of ischemia, of neurodegenerative diseases, of memory and attention disorders, and in identifying compounds which may be agonists, antagonists and/or inhibitors which are potentially useful in therapy, and to production of such polypeptides and polynucleotides.

This invention relates to newly identified uses of the human bombesin receptor 3 (BRS3) polypeptides and polynucleotides encoding such polypeptides, to their use in therapy and in identifying compounds which may be agonists, antagonists and/or inhibitors which are potentially useful in therapy, and to production of such polypeptides and polynucleotides.

Bombesin is a 14 amino acid peptide isolated from frog skin. The mammalian counterparts of the frog peptide are neuromedin B (NMB) and gastrin-releasing peptide (GRP), as well as the biologically active GRP fragment neuromedin C (NMC); (Kroog et al (1995) Med. Res. Rev. 15, 389-417; Smart, D. (1998) in The RBI Handbook of Receptor Classification and Signal Transduction (Ed. K. J. Watling), pp 72-73). Four bombesin receptors have been identified and cloned in vertebrates (Smart, D.(1998) supra). The first two receptors to be cloned were BB₁ and BB₂, which were originally referred to as the NMB- and GRP-preferring bombesin receptors, after their respective endogenous ligands (Benya et al (1995) Mol. Pharmacol. 42, 1058-1068). More recently a third mammalian bombesin receptor subtype, BB₃ (also known as BRS3), was cloned (Fathi et al (1993) J. Biol. Chem. 268, 5979-5984), but no endogenous ligand has been identified to date (Smart, D.(1998) supra). A fourth bombesin receptor, BB₄, has been cloned from the frog. All the receptor subtypes studied couple via G_{q} to the phospholipase C signalling pathway (Kroog et al (1995) supra; Smart, D.(1998) supra).

Bombesin receptor pharmacology was originally defined in terms of the rank order of potency of the endogenous ligands NMB, GRP and bombesin, with the rank order of potency being NMB≧bombesin>GRP at the BB₁ receptor and GRP≧bombesin>NMB at the BB₂ receptor (Kroog et al (1995) supra). Further characterisation became possible with the development of a range of peptide GRP antagonists, most notably BW 1023U90 and (D-Phe⁶ DesMet¹⁴) bombesin 6-14 ethylamide, although it should be noted that the latter is also a partial agonist at the BB₁ receptor (Smart, D.(1998) supra). More recently a non-peptide BB₁ selective PD 165929 has been reported, which has an affinity of 7.6 nM at the BB₁ receptor, but is inactive at the BB₂ receptor (Smart, D.(1998) supra).

Bombesin receptors play a role in a variety of physiological systems in mammals, although the precise subtype involved is, at present, often ill defined. Bombesin receptors are widely distributed in the CNS, as well as in the oesophagus, the lungs, the gastro-intestinal (GI) tract and the reproductive organs (Kroog et al (1995) supra). In the CNS bombesin-like peptides induce satiety, possibly via an interaction with corticotrophin-releasing factor (Smart, D.(1998) supra). They also play a role in thermoregulation, induce histamine-independent pruritus and act at the suprachiasmatic and dorsal raphe nuclei to regulate circadian rhythms and the activity of the 5-HT system, resulting in an association with sleep disorders and depression (Kroog et al (1995) supra; Smart, D.(1998) supra). In the GI tract these peptides regulate smooth muscle contractility, pancreatic secretion, and the release of other peptides, e.g. gastrin. Bombesin-like peptides also have a developmental role in the lung and the uterus, acting as potent growth factors for both normal and neoplastic tissues, and are associated with small cell lung cancers in particular (Kroog et al (1995) supra; Smart, D.(1998) supra). Finally, these peptides stimulate natural killer cell activity and chemotaxis, as well as inducing the release of thyroptropin (Kroog et al (1995) supra; Smart, D.(1998) supra). More recently, using knockout stratergies, it has been shown that mice lacking either the BB₁ or BB₂ receptor do not display gross phenotypical changes compared to the wild type, whilst the BB₃ receptor knockouts have an obese phenotype (Ohki-Hamazaki et al (1997) Nature, 390, 165-169).

The present invention is based on the finding that activation of the BRS3 receptor is strongly neuroprotective. Neuroprotection is a major goal of the pharmaceutical industry for the prevention and/or treatment of diseases such as stroke, ischaemia, head injury, Alzheimer's disease, and also learning, memory and attention disorders.

Thus the present invention provides the use of a compound selected from:

(a) a BRS3 polypeptide;

(b) a compound which activates a BRS3 polypeptide;

(c) a compound which inhibits a BRS3 polypeptide; or

(d) a polynucleotide encoding a BRS3 polypeptide, for the manufacture of a medicament for treating:

(i) stroke;

(ii) ischaemia;

(iii) head injury;

(iv) Alzheimer's disease;

(v) Parkinson's disease;

(vi) learning; or

(vii) memory and attention disorders.

In a first aspect, the present invention relates to the use of BRS3polypeptides. Such polypeptides include:

a) an isolated polypeptide comprising an amino acid sequence having at least a 95% identity, most preferably at least a 97-99% identity, to that of SEQ ID NO:2 over the entire length of SEQ ID NO:2;

b) an isolated polypeptide comprising the amino acid of SEQ ID NO:2;

c) the isolated polypeptide of SEQ ID NO:2;

d) an isolated polypeptide encoded by a polynucleotide comprising a polynucleotide having at least 95% identity to the polynucleotide sequence of SEQ ID NO:1;

e) an isolated polypeptide encoded by a polynucleotide comprising a polynucleotide having the sequence of SEQ ID NO:1.

In addition the polypeptides of the invention include variants and fragments and portions of such polypeptides in (a) to (e) that generally contain at least 30 amino acids, more preferably at least 50 amino acids, thereof.

The polypeptides of the present invention may be in the form of the “mature” protein or may be a part of a larger protein such as a fusion protein. It is often advantageous to include an additional amino acid sequence which contains secretory or leader sequences, pro-sequences, sequences which aid in purification such as multiple histidine residues, or an additional sequence for stability during recombinant production.

The present invention also includes include variants of the aforementioned polypeptides, that is polypeptides that vary from the referents by conservative amino acid substitutions, whereby a residue is substituted by another with like characteristics. Typical such substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10, 1-5, 1-3, 1-2 or 1 amino acids are substituted, deleted, or added in any combination.

Also preferred are biologically active fragments that mediate activities ofBRS3, including those with a similar activity or an improved activity, or with a decreased undesirable activity. Also included are those fragments that are antigenic or immunogenic in an animal, especially in a human. Particularly preferred are fragments comprising receptors or domains of enzymes that confer a function essential to initiate, or maintain cause the Diseases in an individual, particularly a human.

Fragments of the polypeptides of the invention may be employed for producing the corresponding full-length polypeptide by peptide synthesis; therefore, these variants may be employed as intermediates for producing the full-length polypeptides of the invention.

The polypeptides of the present invention may be in the form of a “mature” protein or may be a part of a larger protein such as a fusion protein. It is often advantageous to include an additional amino acid sequence that contains secretory or leader sequences, pro-sequences, sequences that aid in purification, for instance, multiple histidine residues, or an additional sequence for stability during recombinant production.

The present invention also includes variants of the aforementioned polypeptides, that is polypeptides that vary from the referents by conservative amino acid substitutions, whereby a residue is substituted by another with like characteristics. Typical substitutions are among Ala, Val, Leu and Ile; among Ser and Thr, among the acidic residues Asp and Glu; among Asn and Gin; and among the basic residues Lys and Arg; or aromatic residues Phe and Tyr. Particularly preferred are variants in which several, 5-10, 1-5, 1-3, 1-2 or 1 amino acids are substituted, deleted, or added in any combination.

Polypeptides of the present invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

In a further aspect, the present invention relates to BRS3 polynucleotides. Such polynucleotides include isolated polynucleotides comprising a nucleotide sequence encoding a polypeptide which has at least 95% identity, preferably at least 97-99%, to the amino acid sequence of SEQ ID NO:2, over the entire length of SEQ ID NO:2. In this regard, polypeptides which have at least 97% identity are highly preferred, whilst those with at least 98-99% identity are more highly preferred, and those with at least 99% identity are most highly preferred. Such polynucleotides include a polynucleotide comprising the nucleotide sequence. contained in SEQ ID NO:1 encoding the polypeptide of SEQ ID NO:2.

Further polynucleotides of the present invention include isolated polynucleotides comprising a nucleotide sequence that has at least 95% identity, to a nucleotide sequence encoding a polypeptide of SEQ ID NO:2, over the entire coding region. In this regard, polynucleotides which have at least 97% identity are highly preferred, whilst those with at least 98-99% identity are more highly preferred, and those with at least 99% identity are most highly preferred.

Further polynucleotides of the present invention include isolated polynucleotides comprising a nucleotide sequence which has at least 95% identity, to SEQ ID NO:1 over the entire length of SEQ ID NO:1, and variants of these polynucleotides which differ in terms of the 3′-untranslated region. In this regard, polynucleotides which have at least 97% identity are highly preferred, whilst those with at least 98-99% identiy are more highly preferred, and those with at least 99% identity are most highly preferred. Such polynucleotides include a polynucleotide comprising the polynucleotide of SEQ ID NO:1 as well as the polynucleotide of SEQ ID NO:1. The polynucleotides of the invention further include those encoding the human BRS3.

The invention also provides polynucleotides which are complementary to all the above described polynucleotides.

The nucleotide sequence of SEQ ID NO:1 is a cDNA sequence encoding the human BRS3 polypeptide of SEQ ID NO:2. The polypeptide of SEQ ID NO:2 is almost identical to the BRS3 peptide sequence deposited in GenBank (GenBank Acc. No: Z97632) with the exception of an alanine at position 39, whereas Z97632 has a threonine at this position. The nucleotide sequence encoding the polypeptide of SEQ ID NO:2 may be identical to the polypeptide encoding sequence contained in SEQ ID NO:1 or it may be a sequence other than the one contained in SEQ ID NO:1, which, as a result of the redundancy (degeneracy) of the genetic code, also encodes the polypeptide of SEQ ID NO:2. The BRS3 receptor is 399 amino acids in length which has approximately 50% homology with the BB1 and BB2 receptors. BRS3 is encoded by a gene located on the X chromosome. BRS3 has a distinct tissue distribution, being found in the hypothalamus, especially the PVN, ARC, VMH and preoptic areas, as well as the brain stem, amygdala and spinal cord. It has also been identified in secondary spermatocytes, bronchial epithelial cells & the uterus, as well as in small cell lung, breast and epidermal carcinomas. The natural ligand has yet to be identified, with all known natural bombesin-like peptides, including GRP, NMB and NMC, having very low (high μM) affinity. However, some GRP antagonists have moderate (low μM) affinity at BRS3, with the most potent being [DPhe⁶, βAla¹¹, Phe¹³, Nle¹⁴]Bombesin(6-14), which has a low nM affinity. However, this surrogate ligand has similar affinities at the BB1 and BB2 receptors. Activation of BRS3 leads to a phospholipase C mediated mobilisation of intracellular calcium, as well as increased phospholipase D and tyrosine kinase activity. However, little is known about the function of BRS3, as there are currently no selective tools and only one in vivo study has been published.

BRS3 polynucleotides of the present invention may be obtained, using standard cloning and screening techniques, from a cDNA library derived from mRNA in cells ofbrain, lung, spinal cord, pituitary, intestine, placenta, testes or uterus, using techniques well established in the art (for example Sambrook et al., Molecular Cloning: A Laboratory Manual, 2nd Ed., Cold Spring Harbor Laboratory Press, Cold Spring Harbor, N.Y. (1989). Polynucleotides of the invention can also be obtained from natural sources such as genomic DNA libraries or can be synthesized using well known and commercially available techniques.

Recombinant polypeptides of the present invention may be prepared by processes well known in the art from genetically engineered host cells comprising expression systems. Accordingly, in a further aspect, the present invention relates to expression systems which comprise a polynucleotide or polynucleotides of the present invention, to host cells which are genetically engineered with such expression sytems and to the production of polypeptides of the invention by recombinant techniques. Cell-free translation systems can also be employed to produce such proteins using RNAs derived from the DNA constructs of the present invention.

For recombinant production, host cells can be genetically engineered to incorporate expression systems or portions thereof for polynucleotides of the present invention. Introduction of polynucleotides into host cells can be effected by methods described in many standard laboratory manuals, such as Davis et al., Basic Methods in Molecular Biology (1986) and Sambrook et al., Molecular Cloning: A Laboratory Manual, 2nd Ed., Cold Spring Harbor Laboratory Press, Cold Spring Harbor, N.Y. (1989). Preferred such methods include, for instance, calcium phosphate transfection, DEAE-dextran mediated transfection, transvection, microinjection, cationic lipid-mediated transfection, electroporation, transduction, scrape loading, ballistic introduction or infection.

Representative examples of appropriate hosts include bacterial cells, such asStreptococci, Staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

A great variety of expression systems can be used, for instance, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector which is able to maintain, propagate or express a polynucleotide to produce a polypeptide in a host may be used. The appropriate nucleotide sequence may be inserted into an expression system by any of a variety of well-known and routine techniques, such as, for example, those set forth in Sambrook et al., MOLECULAR CLONING, A LABORATORY MANUAL (supra). Appropriate secretion signals may be incorporated into the desired polypeptide to allow secretion of the translated protein into the lumen of the endoplasmic reticulum, the periplasmic space or the extracellular environment. These signals may be endogenous to the polypeptide or they may be heterologous signals.

If a polypeptide of the present invention is to be expressed for use in screening assays, it is generally preferred that the polypeptide be produced at the surface of the cell. In this event, the cells may be harvested prior to use in the screening assay. If the polypeptide is secreted into the medium, the medium can be recovered in order to recover and purify the polypeptide. If produced intracellularly, the cells must first be lysed before the polypeptide is recovered.

Polypeptides of the present invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding proteins may be employed to regenerate active conformation when the polypeptide is denatured during isolation and/or purification.

Polypeptides of the present invention are responsible for one or more biological functions, including one or more disease states, in particular the Diseases hereinbefore mentioned. It is therefore desirous to devise screening methods to identify compounds which stimulate or which inhibit the function of the polypeptide. Accordingly, in a further aspect, the present invention provides for a method of screening compounds to identify those which stimulate or which inhibit the function of the polypeptide. In general, agonists or antagonists, preferably agonists may be employed for therapeutic and prophylactic purposes for such Diseases as hereinbefore mentioned. Compounds may be identified from a variety of sources, for example, cells, cell-free preparations, chemical libraries, and natural product mixtures. Such agonists, antagonists or inhibitors so-identified may be natural or modified substrates, ligands, receptors, enzymes, etc., as the case may be, of the polypeptide; or may be structural or functional mimetics thereof (see Coliganet al., Current Protocols in Immunology 1(2):Chapter 5 (1991)).

The screening method may simply measure the binding of a candidate compound to the polypeptide, or to cells or membranes bearing the polypeptide, or a fusion protein thereof by means of a label directly or indirectly associated with the candidate compound. Alternatively, the screening method may involve competition with a labeled competitor. Such labelled competitors include known BRS3 agonists, for example [D-Phe⁶, βAla¹¹, Phe¹³, Nle¹⁴]Bombesin(6-14) and [D-Tyr⁶, βAla¹¹, Phe¹³, Nle¹⁴]Bombesin(6-14). Further, these screening methods may test whether the candidate compound results in a signal generated by activation or inhibition of the polypeptide, using detection systems appropriate to the cells bearing the polypeptide. Inhibitors of activation are generally assayed in the presence of a known agonist and the effect on activation by the agonist by the presence of the candidate compound is observed. Constitutively active polpypeptides may be employed in screening methods for inverse agonists or inhibitors, in the absence of an agonist or inhibitor, by testing whether the candidate compound results in inhibition of activation of the polypeptide. Further, the screening methods may simply comprise the steps of mixing a candidate compound with a solution containing a polypeptide of the present invention, to form a mixture, measuring BRS3 activity in the mixture, and comparing the BRS3 activity of the mixture to a standard. Fusion proteins, such as those made from Fc portion and BRS3 polypeptide, as hereinbefore described, can also be used for high-throughput screening assays to identify antagonists for the polypeptide of the present invention (see D. Bennett et al., J Mol Recognition, 8:52-58 (1995); and K. Johanson et al., J Biol Chem, 270(16):9459-9471 (1995)).

The polynucleotides, polypeptides and antibodies to the polypeptide of the present invention may also be used to configure screening methods for detecting the effect of added compounds on the production of mRNA and polypeptide in cells. For example, an ELISA assay may be constructed for measuring secreted or cell associated levels of polypeptide using monoclonal and polyclonal antibodies by standard methods known in the art. This can be used to discover agents which may inhibit or enhance the production of polypeptide(also called antagonist or agonist, respectively) from suitably manipulated cells or tissues.

The polypeptide may be used to identify membrane bound or soluble receptors, if any, through standard receptor binding techniques known in the art. These include, but are not limited to, ligand binding and crosslinking assays in which the polypeptide is labeled with a radioactive isotope (for instance, ¹²⁵I), chemically modified (for instance, biotinylated), or fused to a peptide sequence suitable for detection or purification, and incubated with a source of the putative receptor (cells, cell membranes, cell supernatants, tissue extracts, bodily fluids). Other methods include biophysical techniques such as surface plasmon resonance and spectroscopy. These screening methods may also be used to identify agonists and antagonists of the polypeptide which compete with the binding of the polypeptide to its receptors, if any. Standard methods for conducting such assays are well understood in the art.

Examples of potential polypeptide antagonists include antibodies or, in some cases, oligonucleotides or proteins which are closely related to the ligands, substrates, receptors, enzymes, etc., as the case may be, of the polypeptide, e.g., a fragment of the ligands, substrates, receptors, enzymes, etc.; or small molecules which bind to the polypetide of the present invention but do not elicit a response, so that the activity of the polypeptide is prevented.

Thus, in another aspect, the present invention relates to a screening kit for identifying agonists, antagonists, ligands, receptors, substrates, enzymes, etc. for polypeptides of the present invention; or compounds which decrease or enhance the production of such polypeptides, which comprises:

(a) a polypeptide of the present invention;

(b) a recombinant cell expressing a polypeptide of the present invention;

(c) a cell membrane expressing a polypeptide of the present invention; or

(d) antibody to a polypeptide of the present invention;

which polypeptide is preferably that of SEQ ID NO:2.

It will be appreciated that in any such kit, (a), (b), (c) or (d) may comprise a substantial component.

It will be readily appreciated by the skilled artisan that a polypeptide of the present invention may also be used in a method for the structure-based design of an agonist, antagonist or inhibitor of the polypeptide, by:

(a) determining in the first instance the three-dimensional structure of the polypeptide;

(b) deducing the three-dimensional structure for the likely reactive or binding site(s) of an agonist, antagonist or inhibitor;

(c) synthesing candidate compounds that are predicted to bind to or react with the deduced binding or reactive site; and

(d) testing whether the candidate compounds are indeed agonists, antagonists or inhibitors.

It will be further appreciated that this will normally be an interative process.

In a further aspect, the present invention provides methods of treating abnormal conditions such as, for instance, stroke, ischaemia, head injury, Alzheimer's disease, Parkinson's disease, learning or memory and attention disorders, related to either an excess of, or an under-expression of, BRS3 polypeptide activity.

If the activity of the polypeptide is in excess, several approaches are available. One approach comprises administering to a subject in need thereof an inhibitor compound (antagonist) as hereinabove described, optionally in combination with a pharmaceutically acceptable carrier, in an amount effective to inhibit the function of the polypeptide, such as, for example, by blocking the binding of ligands, substrates, receptors, enzymes, etc., or by inhibiting a second signal, and thereby alleviating the abnormal condition, In another approach, soluble forms of the polypeptides still capable of binding the ligand, substrate, enzymes, receptors, etc. in competition with endogenous polypeptide may be administered. Typical examples of such competitors include fragments of the BRS3 polypeptide.

In still another approach, expression of the gene encoding endogenous BRS3 polypeptide can be inhibited using expression blocking techniques. Known such techniques involve the use of antisense sequences, either internally generated or externally administered (see, for example, O'Connor, J Neurochem (1991) 56:560 in Oligodeoxynucleotides as Antisense Inhibitors of Gene Expression, CRC Press, Boca Raton, Fla. (1988)). Alternatively, oligonucleotides which form triple helices (“triplexes”) with the gene can be supplied (see, for example, Lee et al., Nucleic Acids Res (1979) 6:3073; Cooney et al., Science (1988) 241:456; Dervan et al., Science (1991) 251:1360). These oligomers can be administered per se or the relevant oligomers can be expressed in vivo. Synthetic antisense or triplex oligonucleotides may comprise modified bases or modified backbones. Examples of the latter include methylphosphonate, phosphorothioate or peptide nucleic acid backbones. Such backbones are incorporated in the antisense or triplex oligonucleotide in order to provide protection from degradation by nucleases and are well known in the art. Antisense and triplex molecules synthesised with these or other modified backbones also form part of the present invention.

In addition, expression of the human BRS3 polypeptide may be prevented by using ribozymes specific to the human BRS3 mRNA sequence. Ribozymes are catalytically active RNAs that can be natural or synthetic (see for example Usman, N, et al., Curr. Opin. Struct. Biol (1996) 6(4), 527-33.) Synthetic ribozymes can be designed to specifically cleave human BRS3 mRNAs at selected positions thereby preventing translation of the human BRS3 mRNAs into functional polypeptide. Ribozymes may be synthesised with a natural ribose phosphate backbone and natural bases, as normally found in RNA molecules. Alternatively the ribosymes may be synthesised with non-natural backbones to provide protection from ribonuclease degradation, for example, 2′-O-methyl RNA, and may contain modified bases.

For treating abnormal conditions related to an under-expression ofBRS3 and its activity, several approaches are also available. One approach comprises administering to a subject a therapeutically effective amount of a compound which activates a polypeptide of the present invention, i.e., an agonist as described above, in combination with a pharmaceutically acceptable carrier, to thereby alleviate the abnormal condition. Examples of such agonists include[D-Phe⁶, βAla¹¹, Phe¹³, Nle¹⁴]Bombesin(6-14) and [D-Tyr⁶, βAla¹¹, Phe¹³, Nle¹⁴]Bombesin(6-14). Alternatively, gene therapy may be employed to effect the endogenous production ofBRS3 by the relevant cells in the subject. For example, a polynucleotide of the invention may be engineered for expression in a replication defective retroviral vector, as discussed above. The retroviral expression construct may then be isolated and introduced into a packaging cell transduced with a retroviral plasmid vector containing RNA encoding a polypeptide of the present invention such that the packaging cell now produces infectious viral particles containing the gene of interest. These producer cells may be administered to a subject for engineering cells in vivo and expression of the polypeptide in vivo. For an overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd (1996). Another approach is to administer a therapeutic amount of a polypeptide of the present invention in combination with a suitable pharmaceutical carrier.

In a further aspect, the present invention provides for pharmaceutical compositions comprising a therapeutically effective amount of a polypeptide, such as the soluble form of a polypeptide of the present invention, agonist/antagonist peptide or small molecule compound, in combination with a pharmaceutically acceptable carrier or excipient. Such carriers include, but are not limited to, saline, buffered saline, dextrose, water, glycerol, ethanol, and combinations thereof. The invention further relates to pharmaceutical packs and kits comprising one or more containers filled with one or more of the ingredients of the aforementioned compositions of the invention. Polypeptides and other compounds of the present invention may be employed alone or in conjunction with other compounds, such as therapeutic compounds.

The composition will be adapted to the route of administration, for instance by a systemic or an oral route. Preferred forms of systemic administration include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if a polypeptide or other compounds of the present invention can be formulated in an enteric or an encapsulated formulation, oral administration may also be possible. Administration of these compounds may also be topical and/or localized, in the form of salves, pastes, gels, and the like.

The dosage range required depends on the choice of peptide or other compounds of the present invention, the route of administration, the nature of the formulation, the nature of the subject's condition, and the judgment of the attending practitioner. Suitable dosages, however, are in the range of 0.1-100 μg/kg of subject. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

Polypeptides used in treatment can also be generated endogenously in the subject, in treatment modalities often referred to as “gene therapy” as described above. Thus, for example, cells from a subject may be engineered with a polynucleotide, such as a DNA or RNA, to encode a polypeptide ex vivo, and for example, by the use of a retroviral plasmid vector. The cells are then introduced into the subject

The following definitions are provided to facilitate understanding of certain terms used frequently herein.

“Allele” refers to one or more alternative forms of a gene occurring at a given locus in the genome.

“Fragment” of a polypeptide sequence refers to a polypeptide sequence that is shorter than the reference sequence but that retains essentially the same biological function or activity as the reference polypeptide. “Fragment” of a polynucleotide sequence refers to a polynucloetide sequence that is shorter than the reference sequence of SEQ ID NO: 1.

“Fusion protein” refers to a protein encoded by two, often unrelated, fused genes or fragments thereof. In one example, EP-A-0 464 discloses fusion proteins comprising various portions of constant region of immunoglobulin molecules together with another human protein or part thereof. In many cases, employing an immunoglobulin Fc region as a part of a fusion protein is advantageous for use in therapy and diagnosis resulting in, for example, improved pharmacokinetic properties [see, e.g., EP-A 0232 262]. On the other hand, for some uses, it would be desirable to be able to delete the Fc part after the fusion protein has been expressed, detected, and purified.

“Homolog” is a generic term used in the art to indicate a polynucleotide or polypeptide sequence possessing a high degree of sequence relatedness to a reference sequence. Such relatedness may be quantified by determining the degree of identity and/or similarity between the two sequences as hereinbefore defined. Falling within this generic term are the terms, “ortholog”, and “paralog”. “Ortholog” refers to polynucleotides/genes or polypeptide that are homologs via speciation, that is closely related and assumed to have commend descent based on structural and functional considerations. “Paralog” refers to polynucleotides/genes or polypeptide that are homologs via gene duplication, for instance, duplicated variants within a genome.

“Identity” reflects a relationship between two or more polypeptide sequences or two or more polynucleotide sequences, determined by comparing the sequences. In general, identity refers to an exact nucleotide to nucleotide or amino acid to amino acid correspondence of the two polynucleotide or two polypeptide sequences, respectively, over the length of the sequences being compared. For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

“Similarity” is a further, more sophisticated measure of the relationship between two polypeptide sequences. In general, “similarity” means a comparison between the amino acids of two polypeptide chains, on a residue by residue basis, taking into account not only exact correspondences between a between pairs of residues, one from each of the sequences being compared (as for identity) but also, where there is not an exact correspondence, whether, on an evolutionary basis, one residue is a likely substitute for the other. This likelihood has an associated ‘score’ from which the “% similarity” of the two sequences can then be determined.

Methods for comparing the identity and similarity of two or more sequences are well known in the art. Thus for instance, programs available in the Wisconsin Sequence Analysis Package, version 9.1 (Devereux J., et al, Nucleic Acids Res, 12, 387-395, 1984, available from Genetics Computer Group, Madison, Wis., USA), for example the programs BESTFIT and GAP, may be used to determine the % identity between two polynucleotides and the % identity and the % similarity between two polypeptide sequences. BESTFIT uses the “local homology” algorithm of Smith and Waterman (J. Mol. Biol., 147:195-197, 1981, Advances in Applied Mathematics, 2, 482-489, 1981) and finds the best single region of similarity between two sequences. BESTFIT is more suited to comparing two polynucleotide or two polypeptide sequences that are dissimilar in length, the program assuming that the shorter sequence represents a portion of the longer. In comparison, GAP aligns two sequences, finding a “maximum similarity”, according to the algorithm of Neddleman and Wunsch (J. Mo.l Biol., 48, 443-453, 1970). GAP is more suited to comparing sequences that are approximately the same length and an alignment is expected over the entire length. Preferably, the parameters “Gap Weight” and “Length Weight” used in each program are 50 and 3, for polynucleotide sequences and 12 and 4 for polypeptide sequences, respectively. Preferably, % identities and similarities are determined when the two sequences being compared are optimally aligned.

Other programs for determining identity and/or similarity between sequences are also known in the art, for instance the BLAST family of programs (Altschul S. F., et al., J. Mol. Biol., 215, 403-410, 1990, Altschul S. F., et al., Nucleic Acids Res., 25:389-3402, 1997, available from the National Center for Biotechnology Information (NCBI), Bethesda, Md., USA and accessible through the home page of the NCBI at www.ncbi.nlm.nih.gov) and FASTA (Pearson W R, Methods in Enzymology, 183: 63-99 (1990); Pearson W R and Lipman D. J., Proc Nat Acad Sci USA, 85: 2444-2448 (1988) (available as part of the Wisconsin Sequence Analysis Package).

Preferably, the BLOSUM62 amino acid substitution matrix (Henikoff S. and Henikoff J. G., Proc. Nat. Acad Sci. USA, 89: 10915-10919 (1992)) is used in polypeptide sequence comparisons including where nucleotide sequences are first translated into amino acid sequences before comparison.

Preferably, the program BESTFIT is used to determine the % identity of a query polynucleotide or a polypeptide sequence with respect to a polynucleotide or a polypeptide sequence of the present invention, the query and the reference sequence being optimally aligned and the parameters of the program set at the default value, as hereinbefore described. Alternatively, for instance, for the purposes of interpreting the scope of a claim including mention of a “% identity” to a reference polynucleotide, a polynucleotide sequence having, for example, at least 95% identity to a reference polynucleotide sequence is identical to the reference sequence except that the polynucleotide sequence may include up to five point mutations per each 100 nucleotides of the reference sequence. Such point mutations are selected from the group consisting of at least one nucleotide deletion, substitution, including transition and transversion, or insertion. These point mutations may occur at the 5′ or 3′ terminal positions of the reference polynucleotide sequence or anywhere between these terminal positions, interspersed either individually among the nucleotides in the reference sequence or in one or more contiguous groups within the reference sequence. In other words, to obtain a polynucleotide sequence having at least 95% identity to a reference polynucleotide sequence, up to 5% of the nucleotides of the in the reference sequence may be deleted, substituted or inserted, or any combination thereof, as hereinbefore described. The same applies mutatis mutandis for other % identities such as 96%, 97%, 98%, 99% and 100%.

For the purposes of interpreting the scope of a claim including mention of a “% identity” to a reference polypeptide, a polypeptide sequence having, for example, at least 95% identity to a reference polypeptide sequence is identical to the reference sequence except that the polypeptide sequence may include up to five point mutations per each 100 amino acids of the reference sequence. Such point mutations are selected from the group consisting of at least one amino acid deletion, substitution, including conservative and non-conservative substitution, or insertion. These point mutations may occur at the amino- or carboxy-terminal positions of the reference polypeptide sequence or anywhere between these terminal positions, interspersed either individually among the amino acids in the reference sequence or in one or more contiguous groups within the reference sequence. In other words, to obtain a sequence polypeptide sequence having at least 95% identity to a reference polypeptide sequence, up to 5% of the amino acids of the in the reference sequence may be deleted, substituted or inserted, or any combination thereof, as hereinbefore described. The same applies mutatis mutandis for other % identities such as 96%, 97%, 98%, 99%, and 100%.

A preferred meaning for “identity” for polynucleotides and polypeptides, as the case may be, are provided in (1) and (2) below.

(1) Polynucleotide embodiments further include an isolated polynucleotide comprising a polynucleotide sequence having at least a 95, 97 or 100% identity to the reference sequence of SEQ ID NO:1, wherein said polynucleotide sequence may be identical to the reference sequence of SEQ ID NO:1 or may include up to a certain integer number of nucleotide alterations as compared to the reference sequence, wherein said alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence, and wherein said number of nucleotide alterations is determined by multiplying the total number of nucleotides in SEQ ID NO:1 by the integer defining the percent identity divided by 100 and then subtracting that product from said total number of nucleotides in SEQ ID NO:1, or:

nₙ≦xₙ−(xₙ·y),

wherein nₙ is the number of nucleotide alterations, xₙ is the total number of nucleotides in SEQ ID NO:1, y is 0.95 for 95%, 0.97 for 97% or 1.00 for 100%, and · is the symbol for the multiplication operator, and wherein any non-integer product of xₙ and y is rounded down to the nearest integer prior to subtracting it from xₙ. Alterations of a polynucleotide sequence encoding the polypeptide of SEQ ID NO:2 may create nonsense, missense or frameshift mutations in this coding sequence and thereby alter the polypeptide encoded by the polynucleotide following such alterations.

(2) Polypeptide embodiments further include an isolated polypeptide comprising a polypeptide having at least a 95, 97 or 100% identity to a polypeptide reference sequence of SEQ ID NO:2, wherein said polypeptide sequence may be identical to the reference sequence of SEQ ID NO:2 or may include up to a certain integer number of amino acid alterations as compared to the reference sequence, wherein said alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence, and wherein said number of amino acid alterations is determined by multiplying the total number of amino acids in SEQ ID NO:2 by the integer defining the percent identity divided by 100 and then subtracting that product from said total number of amino acids in SEQ ID NO:2, or:

nₐ≦xₐ−(xₐ·y),

wherein nₐ is the number of amino acid alterations, xₐ is the total number of amino acids in SEQ ID NO:2, y is 0.95 for 95%, 0.97 for 97% or 1.00 for 100%, and · is the symbol for the multiplication operator, and wherein any non-integer product of xₐ and y is rounded down to the nearest integer prior to subtracting it from xₐ.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Moreover, a polynucleotide or polypeptide that is introduced into an organism by transformation, genetic manipulation or by any other recombinant method is “isolated” even if it is still present in said organism, which organism may be living or non-living.

“Splice Variant” as used herein refers to cDNA molecules produced from RNA molecules initially transcribed from the same genomic DNA sequence but which have undergone alternative RNA splicing. Alternative RNA splicing occurs when a primary RNA transcript undergoes splicing, generally for the removal of introns, which results in the production of more than one mRNA molecule each of that may encode different amino acid sequences. The tern splice variant also refers to the proteins encoded by the above cDNA molecules.

“Polynucleotide” generally refers to any polyribonucleotide or polydeoxribonucleotide, which may be unmodified RNA or DNA or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs comprising one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may comprise amino acids other than the 20 gene-encoded amino acids. “Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may comprise many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (see, for instance, PROTEINS—STRUCTURE AND MOLECULAR PROPERTIES, 2nd Ed., T. E. Creighton, W. H. Freeman and Company, New York, 1993; Wold, F., Post-translational Protein Modifications: Perspectives and Prospects, pgs. 1-12 in POSTTRANSLATIONAL COVALENT MODIFICATION OF PROTEINS, B. C. Johnson, Ed., Academic Press, New York, 1983; Seifter, et al., “Analysis for protein modifications and nonprotein cofactors”, Meth Enzymol. (1990) 182:626-646 and Rattan, et al., “Protein Synthesis: Post-translational Modifications and Aging”, Ann NY Acad Sci (1992) 663:48-62).

“Variant” refers to a polynucleotide or polypeptide that differs from a reference polynucleotide or polypeptide, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

All publications including, but not limited to, patents and patent applications, cited in this specification or to which this patent application claims priority, are herein incorporated by reference as if each individual publication were specifically and individually indicated to be incorporated by reference herein as though fully set forth.

### EXAMPLES

### Example 1

Tissue Localisation by Taqman and In Situ Hybridisation.

Taqman

TaqMan RT-PCR was carried out as previously described (Medhurst et al.(1999) Br. J. Pharmacol. 128:627-636. Human polyA+mRNA samples were obtained from Clontech. OligodT-primed cDNA synthesis was performed in triplicate using 200 ng human polyA+mRNA and Superscript II reverse transcriptase according to manufacturers instructions (Life Technologies). TaqMan PCR assays were performed on cDNA samples or genomic DNA standards in 96-well optical plates on an ABI Prism 7700 Sequence Detection system (PE Applied Biosystems) according to manufacturers instructions. The primer and probe sequences were as follows:—for human BRS-3:

| | |
|---|---|
| sense | 5′-TCCCGAAAGAGAATTGCCAG |
| antisense | 5′-GAGGTGATTTGGCAACCAGC |
| probe | 5′-AGGGCAAACAGAGCCACCAACACC |

for human cyclophilin:

| | |
|---|---|
| sense | 5′-TGAGACAGCAGATAGAGCCAAGC |
| antisense | 5′-TCCCTGCCAATTTGACATCTTC |
| probe | 5′-CATCACCATTGGCAATGAGCGGTTCC |

Data were analysed using the relative standard curve method with each sample being normalised to cyclophilin to correct for differences in RNA quality and quantity (Medhurst et al (1999) supra).

In-Situ Analysis

For in-situ analysis oligonucleotide probes 3′ end labelled with ³⁵S dATP were used. The sequences were as follows:

Sense
5′ CCA TGC AAA CAG TTC CAA ATA TTT TCA TCA 3′
Antisense
5′ TGA TGA AAA TAT TTG GAA CTG TTT GCA TGG 3′

COP purified oligo probes were obtained from Cruachem. 10pmoles of antisense and sense probe were 3′ end labelled with ³⁵S dATP using an NEN terminal transferase end labelling kit. The probes were purified to remove unlabelled probe and unincorporated label using Nensorb purification cartridges.

Slides of brain sections were prepared. The brains had been removed and immediately frozen in isopentane over dry ice. 12 micron sections of brain were cut on a cryostat and fixed in 4% paraformaldehyde. The sections were acetylated in acetic anhydride, dehydrated through a series of alcohols and defatted in chloroform.

One million counts of probe were applied to each slide in 100 ul hybridisation buffer (10% dextran sulphate, 4×SSC, 1×Denhardts solution, 0.1 mg/ml salmon sperm DNA, 0.1 mg/ml tRNA, 0.1 mg/ml polyA, 50% deionised formamide). Cold probe was also included in the hybridisation mix in some instances to assess ability to compete off binding of labelled probe to the mRNA. Sections were incubated overnight at 27 degrees C. Unbound probe was removed by washing in 1×SSC at 52-58 degrees C. for 1 hour, followed by washing with 1×SSC at room temperature for 2 hours. The slides were then rinsed in water and air dried before being placed on phosphor screens for 3 to 4 days or exposed to Xray film for 14 days.

The localisation results from Taqman and in-situ analysis are expressed as relative expression levels, with a scoring system as follows:

+++ is highest expression, ++ expression;
+ low, but detectable expression; − showed no detectable expression:

| | |
|---|---|
| Hippocampus (Layers CA1 to CA3 and dendate gyrus) | +++ |
| Amygdala | +++ |
| Caudate Nucleus | ++ |
| Corpus callosum | ++ |
| Cortex (mainly Layers 4 & 5 of temporal | ++ |
| cortex, adjacent to hippocampus) | |
| Hypothalamus | +++ |
| Putamen | + |
| Substantia Nigra | + |
| Thalamus | + |
| Spinal cord (white & gray matter but not DRG) | + |
| Pituitary gland | ++ |
| Heart | − |
| cerebellum | − |
| liver | − |
| lung | − |

### Example 2

Agonist Pharmacology at the Endogenous Human BRS-3 in NCI-N417 Cells

NCI-N417 cells were seeded into Costar 96 well black walled plates (30,000 cells per well), cultured overnight in RPMI 1640 medium containing 10% foetal calf serum and 1% L-glutamine, and then loaded with the cytoplasmic calcium indicator Fluo-3AM (4 μM) in the presence of 2.5 mM probenecid at 37° C. for 60 min. The cells were then washed 4× with, and finally resuspended in, Tyrode's medium containing 2.5 mM probenecid. Fluorescence was then monitored using a FLIPR (λ_{EX}=488 nm, λ_{EM}=540 nm) before and after the addition of various ligands (10 pM-10 μM), as described previously (Smart et al (1999). Br. J. Pharmacol. 128:1-4).

| | pEG₅₀ | Efficacy |
|---|---|---|
| [D-Phe⁶, βAla¹¹, Phe¹³, | 7.88 ± 0.04 | full |
| Nle¹⁴]BN(6-14) | | |
| NMB | 5.65 ± 0.07 | full |
| NMC | <5 | 20% at 10 μM |
| Ranantensin | 5.59 ± 0.12 | full |
| Bombesin | <5 | 23% at 10 μM |
| PG-L | 5.54 ± 0.05 | full |
| GRP | <5 | 23% at 10 μM |
| [D-Tyr⁶, βAla^{11, Phe}^{13 ,} | 8.02 ± 0.03 | full |
| Nle¹⁴]BN(6-14) | | |
| AcNMB(3-10) | 5.73 ± 0.03 | full |
| [D-Phe¹, Nle⁹]litorin | 5.96 ± 0.05 | full |
| [D-Phe⁶]BN(6-13) | 5.86 ± 0.09 | full |
| propylaniide | | |
| [D-Phe⁶, Phe¹³]BN(6-13) | 6.83 ± 0.03 | full |
| propylamide | | |

### Example 3

Demonstration of Neuroprotective Effects of Activation of BRS3

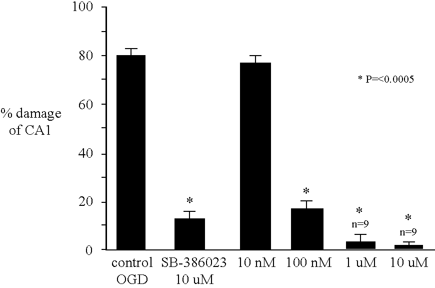

This data was generated using the oxygen/glucose deprivation model (Pringle, et al (1997) Brain Research 755:36-46) in organotypic hippocampal slice cultures (Stoppini et al (1991) J Neurosci. Methods, 37:173-182) using [D-Phe⁶, βAla¹¹, Phe¹³, Nle¹⁴]Bombesin(6-14) and shows that activation of BRS-3 is neuroprotective.

Primary Hippocampal Cell Culture

Hippocampi were isolated from embryonic Sprague Dawley rats (gestational age 17.5 days; Charles River), incubated with 0.08% (w/v) trypsin, and dissociated in Neurobasal medium containing 10% heat-inactivated fetal calf serum (Skaper et al.(1990) Methods in Neurosciences, Vol. 2 (Conn P. M., ed), pp. 17-33 Academic Press, San Diego). Cells were pelleted by centrifugation (200 g, 5 min) and resuspended in Neurobasal medium containing B27 supplements (with antioxidants), 25 μM glutamate, 1 mM sodium pyruvate, 2 mM L-glutamine, 50 U/ml penicillin, and 50 μg/ml streptomycin. The cell suspension was plated onto poly-D-lysine (10 μg/ml) coated 48-well culture plates (Nunc), at a density of 4.5×10⁴ cells per cm². Cultures were maintained at 37° C. in a humidified atmosphere of 5% CO₂-95% air. After 5 days, one-half the medium was replaced with an equal volume of maintenance medium (plating medium but containing B27 supplements without antioxidants, and lacking glutamate). Additional medium exchanges (0.5 volume) were performed every 3-4 days thereafter. Cells were used between 14-16 days in culture. During this period, neurons developed extensive neuritic networks, and formed functional synapses

Neurotoxicity Assays

Cultures were washed once with Locke's solution (pH 7.0-7.4) (Skaper et al. (1990) supra) with or without 1 mM magnesium chloride (MgCl₂). To induce sub-maximal neurotoxicity, cultures were exposed for 15 min at room temperature to MgCl₂-locke's solution, supplemented with 0.1 μM glycine and 30 μM histamine. Thereafter, cells were washed with complete Locke's solution and returned to their original culture medium for 24 h. Cytotoxicity was evident during the 24 h after the insult. Viable neurons had phase-bright somata of round-to-oval shape, with smooth, intact neurites. Neurons were considered nonviable when they exhibited neurite fragmentation and somatic swelling and vacuolation. Cell survival was quantified 24 h after the insult by a colorimetric reaction with 3-(4,5-dimethylthiazol-2-yl)-2,5-diphenyltetrazolium bromide (MTT) (Mosmann (1983) J. Immunol. Methods 65:55-63; Manthorpe et al. (1986) Dev. Brain Res. 25:191-198; Skaper et al., (1990) supra). Absolute MTT values obtained were normalized and expressed as a percentage of sham-treated sister cultures (defined as 100%). Control experiments showed that the loss of viable neurons assessed in this manner was proportional to the number of neurons damaged, as estimated by trypan blue staining.

The results show that in the hippocampal neurones [D-Phe⁶, βAla¹¹, Phe¹³, Nle¹⁴]Bombesin(6-14) was neuroprotective (pEC₅₀ for inhibition of damage of 7.3±0.1). However, in the cerebellar granule cells [D-Phe⁶, βAla¹¹, Phe¹³, Nle¹⁴]Bombesin(6-14) had no effect on glutamate-induced excitotoxicity. This indicates that [D-Phe⁶, βAla¹¹, Phe¹³, Nle¹⁴]Bombesin(6-14) does not block the NMDA or AMPA receptors, but rather acts by inhibiting the release of endogenous glutamate in the hippocampus.

Sequence Information

| SEQ ID NO:1 | |
|---|---|
| 1 | ATGGCTCAAA GGCAGCCTCA CTCACCTAAT CAGACTcTAA TTTCAATCAC |
| 51 | AAATGACACA GAATCATCAA GCTCTGTGGT TTCTAACGAc AACACgAATA |
| 101 | AAGGATGGAG CGGGGACAAC TCTCCAGGAA TAGAAGCATT GTGTGCCATC |
| 151 | TATATTACTT ATGCTGTGAT CATTTCAGTG GGCATCCTTG GAAATGCTAT |
| 201 | TCTCATCAAA GTCTTTTTCA AGACCAAATC CATGCAAACA GTTCCAAATA |
| 251 | TTTTCATCAC CAGCCTGGCT TTTGGAGATC TTTTACTTCT GCTAACTTGT |
| 301 | gTgccAGTGG ATGCAACTCA cTACCTTGCA GAAGGAtGGC TGTTCGGAAG |
| 351 | aAtTGGTTGT AAGGTGCTCT CTTTCATCCG GCTCACTTCT GTTGGTGTGT |
| 401 | CAGTGTTCAC ATTAgCAATT CTCAGCGCTG ACAGATACAA GGCAGTTGTG |
| 451 | AAGCCACTTG AGCGACAGCC CTCCAAtGCC ATCCTGAAgA CTTGTGTAAA |
| 501 | AGCTGGCTGC GTCTGGATCG TGTCTATGAT ATTTGCTCTA CCTGAGGCTA |
| 551 | TATTTTcAAA TGTATACACT TTTCGAGATC CCaATAAAAA TATGACATTT |
| 601 | GAATCATGTA CCTCTTATCC TGTCTCTAAG AAGCTCTTGC AAGAAATACA |
| 651 | TTCTCTGCTG TGCTTCTTAg TGTTCTACAT TATTCCACTC TCTATTATCT |
| 701 | CTGTCTACTA TTCCTTGATT GCTAGGACCC TTTACAAAAG CACCCTGAAC |
| 751 | ATACCTACTG AGGAACAAAG CCATGCCCGT AAGCAGATTG AATCCCGAAA |
| 801 | GAGAATTGCC AGAACGGTAT TGGTGTTGGT GGCTCTGTTT GCCCTCTGCT |
| 851 | GGTTGCCAAA TCACCTCCTG TACCTCTACC ATTCATTCAC TTCTCAAACC |
| 901 | TATGTAGACC CCTCTGCCAT GCATTTCATT TTCACCATTT TCTCTCGGGT |
| 951 | TTTGGCTTTC AGCAATTCTT GCGTAAACCC CTTTGCTCTC TACTGGCTGA |
| 1001 | GCAAAAGCTT CCAGAAGCAT TTTAAAGCTC AGTTGTTCTG TTGCAAGGCG |
| 1051 | GAGCGGCCTG AGCCTCCTGT TGCTGACACC TCTCTTACCA CCCTGGCTGT |
| 1101 | GATGGGAACG GTCCCGGGCA CTGGGAGCAT ACAGATGTCT GAAATTAGTG |
| 1151 | TGACCTCGTT CACTGGGTGT AGTGTGAAGC AGGCAGAGGA CAGATTCTAG |

SEQ ID NO:2
MAQRQPHSPNQTLISITNDTESSSSVVSNDNTNKGWSGDNSPGIEALCAIYITYAVIISVGILGNAILIKVF
PKTKSMQTVPNIFITSLAFGDLLLLLTCVPVDATHYLAEGWLFGRIGCKVLSFIRLTSVGVSVFTLAILSAD
RYKAVVKPLERQPSNAILKTCVKAGCVWIVSMIFALPEAIFSNVYTFRDPNKNMTFESCTSYPVSKKLLQEI
HSLLCFLVFYIIPLSIISVYYSLIARTLYKSTLNIPTEEQSHARKQIESRKRIARTVLVLVALFALCWLPNH
LLYLYESFTSQTYVDPSAMHFIFTIFSRVLAFSNSCVNPFALYWLSKSFQKHFKAQLFCCKABRPEPPVADT
SLTTISAVMGTVPGTGSIQMSEISVTSFTGCSVKQAEDRF

1. The use of a compound selected from:
(a) a BRS3 polypeptide;
(b) a compound which activates a BRS3 polypeptide;
(c) a compound which inhibits a BRS3 polypeptide; or
(d) a polynucleotide encoding a BRS3 polypeptide, for the manufacture of a medicament for treating:
(i) stroke;
(ii) ischaemia;
(iii) head injury;
(iv) Alzheimer's disease;
(v) Parkinson's disease;
(vi) learning; or
(vii) memory and attention disorders.

2. The use according to claim 1 wherein the medicament comprises an isolated polypeptide which comprises a polypeptide having at least 95% identity to the BRS3polypeptide of SEQ ID NO:2.

3. The use according to claim 2 wherein the isolated polypeptide is the BRS3polypeptide of SEQ ID NO:2.

4. The use according to claim 1 wherein the medicament comprises a compound which activates a BRS3polypeptide.

5. The use according to claim 1 wherein the medicament comprises a compound which inhibits a BRS3polypeptide.

6. The use according to claim 1 wherein the medicament comprises a polynucleotide encoding a polypeptide having at least 95% identity with the amino acid sequence of SEQ ID NO:2.

7. The use according to claim 6 wherein the polynucleotide comprises a polynucleotide having at least 95% identity with the polynucleotide of SEQ ID NO:1.

8. The use according to claim 6 or 7 wherein the polynucleotide has the polynucleotide sequence of SEQ ID NO:1.

9. The use according to claim 1 wherein the compound which activates the BRS3 polypeptide is [D-Phe⁶, βAla¹¹, Phe¹³, Nle¹⁴]Bombesin(6-14) or [D-Tyr⁶, βAla¹¹, Phe¹³, Nle¹⁴]Bombesin(6-14).

10. The use of [D-Phe⁶, βAla¹¹, Phe¹³, Nle¹⁴]Bombesin(6-14) or [D-Tyr⁶, βAla¹¹, Phe¹³, Nle¹⁴]Bombesin(6-14) for the manufacture of a medicament for treating:
(i) stroke;
(ii) ischaemia;
(iii) head injury;
(iv) Alzheimer's disease;
(v) Parkinson's disease;
(vi) learning; or
(vii) memory and attention disorders.

11. A method for screening to identify compounds which stimulate or which inhibit the function of the polypeptide as defined in claim 1 which comprises a method selected from the group consisting of:
(a) measuring the binding of a candidate compound to the polypeptide (or to the cells or membranes bearing the polypeptide) or a fusion protein thereof by means of a label directly or indirectly associated with the candidate compound;
(b) measuring the binding of a candidate compound to the polypeptide (or to the cells or membranes bearing the polypeptide) or a fusion protein thereof in the presense of a labeled competitior;
(c) testing whether the candidate compound results in a signal generated by activation or inhibition of the polypeptide, using detection systems appropriate to the cells or cell membranes bearing the polypeptide;
(d) mixing a candidate compound with a solution containing a polypeptide of claim 1, to form a mixture, measuring activity of the polypeptide in the mixture, and comparing the activity of the mixture to a standard; or
(e) detecting the effect of a candidate compound on the production of mRNA encoding said polypeptide and said polypeptide in cells, using for instance, an ELISA assay.

12. A method according to claim 11 where the labelled competitor is a labelled form of [D-Phe⁶, βAla¹¹, Phe¹³, Nle¹⁴]Bombesin(6-14) or [D-Tyr⁶, βAla¹¹, Phe¹³, Nle¹⁴]Bombesin(6-14).
